(19) United States
(12) Patent Application Publication
(10) Pub. No.: US 2004/0258754 A1
(43) Pub. Date: Dec. 23, 2004

(54) Compositions for oral administration of camptothecin and its analogs

(76) Inventors: Valery Alakhov, Baie D'Urfe (CA); Greg Pietrzynski, Montreal (CA); Evgueni Klinski, Kirkland (CA); Kishore Patel, Lasalle (CA); Shengmin Li, Saint-Laurent (CA); Lev E. Bromberg, Swampscott (CA)

Correspondence Address: Patrick H. Higgins, Mathews, Collins, Shepherd & McKay, P.A., Suite 306, 100 Thanet Circle, Princeton, NJ, 08540, US

(21) Appl. No.: 10/464,135

(22) Filed: Jun. 18, 2003

(51) Int. Cl. A61K 9/14; A61K 31/4745

(52) U.S. Cl. 424/486; 514/283

(57) ABSTRACT

Pharmaceutical compositions are provided which comprise a lactone form of camptothecin, or a lactone form analog thereof that is effective for controlling abnormal cell proliferation; and at least one amphiphilic block copolymer that, upon oral administration to a patient, increases oral bioavailability of the lactone form significantly more than that of a directly related carboxylate form of the camptothecin or analog thereof. Compositions are also provided that further comprise at least one bioadhesive polymer that protects a closed alpha-hydroxy lactone structure of the lactone form of camptothecin or analog thereof, and provides a controlled release of the lactone form of camptothecin or lactone form analog thereof, in GI, upon oral administration to a patient. Methods are described for the prevention and/or treatment of a condition related to abnormal cell proliferation comprising orally administering to a patient in need of thereof an effective amount of a composition of the present invention.

### FIELD OF THE INVENTION

The invention relates to compositions and methods for delivering camptothecin and its analogs, such as topotecan and the like, to patients by oral administration as well as methods of treating conditions and disease associated with abnormal cell proliferation such as in cancer.

### BACKGROUND OF THE INVENTION

Camptothecin was originally isolated from the plant, Camptotheca acuminata, in the 1960's (Wall, M. et al. (1966) J. Am. Chem. Soc. 88: 3888-3890). Camptothecin has a pentacyclic ring system with only one asymmetric center in ring E with a 20(S)-configuration. The pentacyclic ring system includes a pyrrole quinoline moiety (rings A, B and C), a conjugated pyridone (ring D), and a six-membered lactone (ring E) with an .alpha.-hydroxyl group (i.e., an .alpha.-hydroxy lactone).

Camptothecin is a highly lipophilic and poorly water-soluble compound. Sodium camptothecin that is solubilized by sodium hydroxide in water was used in clinical trials in the early 70's and found to have antitumor activity. However, this formulation of camptothecin administered via i.v. caused considerable side effects such as myelosuppression and hemorrhagic cystitis.

Further studies of camptothecin revealed that its carboxylate form represents only a minor fraction compared to the native camptothecin with the closed .alpha.-hydroxy lactone ring (Giovanella et al. (1991) Cancer Res. 51:3052). It was also shown that at neutral pH, camptothecin and its derivatives undergo an alkaline hydrolysis of the lactone ring, resulting in a low active carboxylate form of the drug, which represents a major limitation of their therapeutic anticancer efficacy.

Camptothecin and its derivatives have been shown to inhibit DNA topoisomerase I by stabilizing the covalent complex (“cleavable complex”) of enzyme and strand-cleaved DNA. Inhibition of topoisomerase I by camptothecin induces protein-associated DNA single-strandbreaks which occur during the S-phase of the cell cycle. Since the S-phase is relatively short compared to other phases of the cell cycle, longer exposure to camptothecin should result in increased cytotoxicity of tumor cells. Studies indicate that only the closed alpha-hydroxy lactone form of the drug helps stabilize the cleavable complex, leading to inhibition of the cell cycle and apoptosis.

To preserve the alpha-hydroxy lactone form of camptothecin, camptothecin and its water insoluble derivatives have been dissolved in N-methyl-2-pyrrolidinone in the presence of an acid (U.S. Pat. No. 5,859,023). Upon dilution with an acceptable parenteral vehicle, a stable solution of camptothecin was obtained. The concentrated solution of camptothecin was also filled in gel capsules for oral administration. It is believed that such formulations increase the amount of lipophilic alpha-hydroxy lactone form of camptothecin that diffuse through the cellular and nuclear membranes in tumor cells.

T. G. Burke, A. E. Staubus, A. K. Mishra and H. Malak (“Liposomal Stabilization of Camptothecin's Lactone Ring.” J. Am. Chem. Soc. 1992, 114, 8318) and T. G. Burke, A. K. Mishra, M. C. Wani and M. E. Wall (“Lipid bilayer partitioning and stability of camptothecin drugs.” Biochemistry 1993 May 25;32 (20):5352-64) have demonstrated that harboring of camptothecin drugs into phospholipid bilayer membranes stabilized the alpha-hydroxy lactone moiety of camptothecin drugs against hydrolysis. In comparison to hydrolysis half-lives in PBS of approximately 15 to 30 min, lipid bilayer membrane-bound camptothecin drugs were found to be stable even for periods up to 72 hours. These authors have determined an iodide ion induced quenching behavior of camptothecin's fluorescence indicative of intercalation of camptothecin molecules between the phospholipid acyl chains of membrane bilayers, a protected environment removed from the aqueous interface. The potential for stabilization of camptothecin's alpha-hydroxy lactone ring structure in this environment led to the expectation that lipid bilayer intercalation might conserve the biologically active form in vivo, thereby permitting the active form to be delivered via liposomal bilayers into a biological host (U.S. Pat. No. 5,552,156).

It has been reported that in at pH 7.4, human serum albumin (HSA) preferentially binds the carboxylate form of camptothecin with a 150-fold higher affinity than the alpha-hydroxy lactone form (Z Mi and T. G. Burke, Biochemistry 1994 Aug. 30;33 (34):10325-36). These HSA drug interactions lead to a more rapid and complete opening of camptothecin's lactone ring in the presence of HSA compared to the protein free medium. Furthermore, in human plasma, at pH 7.4 and 37° C. the camptothecin's lactone ring opens rapidly and fully leading to the drug conversion into the carboxylate form. In whole blood versus plasma, the camptothecin's lactone was found to display a higher stability, which was found to be due to the drug associations with the lipid bilayers of red blood cells. Nevertheless, camptothecin lactone form still remains thermodynamically and kinetically unstable in the presence of albumin and the concentration of the active alpha-hydroxy lactone form in plasma remains insufficient. Thus, the liposomal bilayers cannot provide a sufficient chemical stability for this drug.

U.S. Pat. Nos. 5,552,156 and 5,736,156 describe liposomes and micelles of surfactant molecules for intravenous delivery of camptothecins. In liposomes, the camptothecin can reside bound to and partially in the membrane interlayer or dissociate into the internal enclosed aqueous layer in direct contact with water where the camptothecin lactone is not stable to hydrolysis. In micelles of surfactant molecules, the camptothecin is either in the central hydrocarbon portion of the micelle, bound to the micelle membrane or bound to the outside of the micelle. However, while camptothecins are less stable in micelles than in liposomes, especially in poly(ethylene oxide)-containing micelles, the amount of camptothecin compound that can bind to the membrane layer in a liposome is limited to the dimensions of the membrane and to the requirement that the membrane remain intact to prevent rupture of the liposome. The ratio of lipid to camptothecin in liposomes is generally greater than 150, and the lactone of the camptothecin slowly hydrolyzes because of the reported equilibrium between bound and free camptothecin.

A method for administering a camptothecin to a patient comprising: injecting into a patient a pharmaceutical composition comprising an aqueous suspension of solid particles suitable for intravenous delivery, the solid particles comprising a camptothecin, and a 0.3 nm to 3.0 mu.m thick layer of a membrane-forming amphipathic lipid was described in U.S. Pat. No. 6,534,080. In this patent the suspensions of micron and submicron size particle coated with membrane forming lipids and/or surfactants at the conditions when the last ones do not form micelles, were shown to significantly reduce deactivation of the camptothecin by hydrolysis in-vitro and by plasma components in-vivo. However, the described compositions are mainly applicable to water insoluble camptothecin derivatives since the amphipathic nature of the lipid components would not allow a sufficient retention of the water soluble drug derivatives such as topotecan. Furthermore, a limited loading capacity of the above compositions produce some dose limitations on the final drug forms.

Oral administration of chemotherapeutic agents is a preferred route of cancer treatment provided that therapeutically significant drug levels can be achieved. However, there are a number of obstacles that considerably limit the efficiency of oral chemotherapy. The most important factors that are directly related to camptothecin and its derivatives include low drug solubility that reduces the surface of its interactions with the gastrointestinal tract (GI), low drug absorption due to the high activity of BCRP, a recently discovered efflux pump that has a high selectivity to camptothecins, and probably, that of some yet unknown drug transporters, as well as high activity in GI of metabolic enzymes such as cytochrome P-450 isoenzymes, leading to a rapid inactivation of the active form of the drug. (for review see, Kruijtzer C M, Beijnen J H, Schellens J H., Oncologist 2002;7(6):516-30)

In addition to a reduced oral bioavailability of camptothecins, a significant intra- and inter-patient variability in the drug absorption related to non equal expression levels and activities of the efflux pumps and metabolic enzymes put a further limitation of the oral administration of these drugs due to a difficulty to control the drug levels within the same dosing regime applied to different patients, or even to the same patient if the treatment is to be repeated at a different time.

Several attempts to overcome the above problems were undertaken recently by using combination therapies with camptothecins, in which these drugs were co-administered with BCRP inhibitors such as GF120918 (Malingre M M. Beijnen J H, Rosing H, Br. J. Cancer, 84:42-47, 2001), Ko143 a new analog of fungal toxin fumitremorgin C (Allen J D, van Loevezijn A, Lakhai J M, van der Valk M, van Tellingen O, Reid G, Schellens J H, Koomen G J, Schinkel A H., Mol Cancer Ther 2002 April;1(6):417-25), and some other compounds with similar properties. As a result, a significant increase in oral absorption of camptothecins and reduction in the intra- and inter-patient variability were achieved. However, this approach did not resolve the low chemical stability problem and similar conversion of the lactone into carboxilate was reported for the oral compositions of camptothecins as for intravenous ones.

Furthermore, significantly higher levels of adverse reactions were reported for chemotherapeutic compositions in which the anticancer drugs were combined with the efflux pump inhibitors due to considerable absorption of these inhibitors into the circulation followed by their interactions with normal tissues (Kruijtzer C M F, Beijnen J H, Rosing H. J. Clin. Oncol. 20:2943-2950, 2002)

Therefore, pharmaceutical compositions are needed for camptothecins wherein the closed alpha-hydroxy lactone forms thereof are stable and soluble under physiological conditions, e.g., in vivo, and high oral bioavailability (e.g., absorption in the gastrointestinal tract) of the therapeutic compounds is provided.

### SUMMARY OF THE INVENTION

The present invention provides water-soluble formulations of camptothecin and its analogs with increased oral bioavailability and higher levels of the lactone form of these compounds in the bloodstream. The present invention also provides methods of manufacturing these formulations, kits containing these formulations and methods of using these formulations to treat patients having diseases associated with abnormal cell proliferation, such as cancer. The present invention is particularly directed to a pharmaceutical composition comprising a lactone form of camptothecin, or a lactone form analog thereof that is effective for controlling abnormal cell proliferation; and at least one amphiphilic block copolymer that, upon oral administration to a patient, increases oral bioavailability of the lactone form significantly more than that of a directly related carboxylate form of the camptothecin or analog thereof. The current invention encompasses compositions wherein at least one amphiphilic block copolymer is selected from the group consisting of poly(ethylene oxide)-b-poly-propylene oxide; poly(ethylene oxide)-alpha tocopherol; poly-ethylene oxide poly-alkyl; and, poly-ethylene oxide poly-lactide. The invention is further directed to compositions wherein at least one amphiphilic block copolymer is poly(ethylene oxide)-b-poly(propylene oxide). Further the invention is directed to pharmaceutical composition comprising a lactone form of camptothecin, or a lactone form analog thereof that is effective for controlling abnormal cell proliferation; an amphiphilic block copolymer that increases oral bioavailability of the lactone form, significantly more than a carboxylate form of the camptothecin or analog thereof; and at least one bioadhesive polymer that protects a closed alpha-hydroxy lactone structure of the lactone form of camptothecin or analog thereof, and provides a controlled release of the lactone form of camptothecin or lactone form analog thereof, in GI, upon oral administration to a patient. The invention is particularly drawn toward these pharmaceutical compositions wherein at least one bioadhesive polymer is a copolymer and at least one bioadhesive segment of the copolymer is polyacrylic acid (PAA), for example, wherein at least one bioadhesive polymer is a copolymer of polyacrylic acid (PAA) and a PEO-PPO-PEO block Poloxamer (polyether). These type compositions are described wherein the copolymer is a graft-copolymer wherein the PAA and the polyether are linked to each other by a plurality of C—C bonds. The invention is further drawn toward methods for the prevention and/or treatment of a condition related to abnormal cell proliferation comprising orally administering to a patient in need of thereof an effective amount of pharmaceutical compositions of the present invention.

### BRIEF DESCRIPTION OF THE FIGURES

FIG. 1 shows a representative chromatogram of CPT-11 (irinotecan) and its metabolites APC and SN38 after equilibration in human serum. As is seen, without encapsulation into gels a substantial portion of CPT-11 is metabolised.

FIG. 2 shows a representative chromatogram of CPT-11 obtained after equilibration of gel based on Pluronic F127-PAA loaded with CPT-11 in human serum for 1 h.

FIG. 3 shows kinetic evaluation of the rate of lactone ring opening for CPT-11 in human serum without gels (filled circles) and with L92-PAA gels (filled diamonds) or with F127-PAA gels (open circles). As is seen, CPT-11 unprotected by the gels was hydrolysed much more rapidly, while the gels proved to be effective barriers against the drug decomposition.

FIG. 4 shows force-distance diagrams obtained for mucoadhesive F127-PAA gel preparations in contact with rat jejunum mucosa at 37° C. As is seen, Pluronic-PAA gels were similar and in many instances exceeded mucoadhesive properties of Carbopol C934P, which is known in the art as an industry standard for mucoadhesive polymers.

### DETAILED DESCRIPTION OF THE INVENTION

Unless defined otherwise, all technical and scientific terms used herein have the same meaning as is commonly understood by one of skill in the art to which this invention belongs. All publications and patents referred to herein are incorporated by reference.

The closed alpha-hydroxy lactone form (lactone form) of camptothecin and its analogs (camptothecins) inhibit the activity of DNA topoisomerase I, thus inhibiting the cell cycle of tumor cells in vivo. The lactone form of these compounds is generally acknowledged to be effective for controlling abnormal cell proliferation and is well-known to exhibit pharmacological efficacy. However, at neutral pH, camptothecin and its derivatives, undergo an alkaline hydrolysis of the lactone ring, which results in the inactivation of the carboxylate form, with the drug having low or no pharmacological efficacy. Accordingly, hydrolysis of the lactone ring of camptothecin and its analogs is a major limitation of anticancer therapeutic efficacy of these compounds.

Oral administration of camptothecins is a preferred route of cancer treatment provided that therapeutically significant drug levels can be achieved. However, problems inherent with camptothecin and its derivatives include low drug solubility under physiological conditions that reduces the surface of its interactions with the gastrointestinal tract (GI) as well as low absorption.

Camptothecins

Camptothecin compounds for use in the present invention preferably contain an intact lactone ring. Camptothecins include, for example, but are not limited to, 9-aminocamptothecin, 7-ethylcamptothecin, 10-hydroxycamptothecin, 9-nitrocamptothecin, 10,11-methlyenedioxycamptothecin, 9-amino-10,1′-methylenedioxycamptothecin or 9-chloro-10,11-methylenedioxycamptothecin, irinotecan (CPT-11), topotecan, (7-(4-methylpiperazinomethylene)-10,11-ethylenedioxy-20(S)-camptothecin, 7-(4-methylpiperazinomethylene)-10,11-methylenedioxy-20(S)-camptothecin or 7-(2-(N-isopropylamino)ethyl)-(20S)-camptothecin.

On May 29, 1996, the FDA approved topotecan, for example, as a treatment for advanced ovarian cancers that have resisted other chemotherapy drugs. Topotecan was demonstrated to exhibit efficacy at least as good as Taxol, for example, in clinical trials. Also, on Jun. 17, 1996, irinotecan, for example, was approved as a treatment for metastatic cancer of the colon or rectum. These compounds are included in “camptothecin compounds” for use in the present invention. Dosages of camptothecin compounds for use in compositions and methods of the present invention are readily determined by those of ordinary skill in the art of medicine and pharmacology. Specific dosage and period of administration will depend particularly on the weight and condition of the subject. See, e.g., O'Leary, et al., Camptothecins: A Review of Their Schedules Of Administration, Eur. J. Cancer., 34(10):1500 (1998); Mathijssen, R. H., Pharmacology Of Topoisomerase I Inhibitors Irinotecan (Cpt-11) And Topotecan, Curr. Cancer Drug Targets, 2(2):103 (2002); Takimoto, C. H., et al., Clinical Applications of the Camptothecins, Biochim. Biophys. Acta., 1400(1-3):107 (1998); and Rodriguez-Galindo, C., et al., Clinical Use of Topoisomerase I Inhibitors In Anticancer Treatment, Med. Pediatr. Oncol., 35(4):385 (2000).

Bioavailability

Pharmaceutical compositions are provided herein for camptothecins wherein the closed alpha-hydroxy lactone forms thereof are stable and soluble under physiological conditions, e.g., in vivo. Moreover, compositions described herein provide high oral bioavailability (e.g., GI absorption) of the camptothecins. Particularly, pharmaceutical compositions of the present invention selectively increases the bioavailability of the camptothecin lactone form compounds upon oral administration to a patient in need of treatment.

Pharmaceutical Compositions

Pharmaceutical compositions are provided herein comprising a closed alpha-hydroxy lactone form of camptothecin, or a closed alpha-hydroxy lactone form analog thereof that is effective for controlling abnormal cell proliferation, and at least one amphiphilic block copolymer that increases the solubility of the lactone form compound under physiological conditions and selectively increases bioavailability of the lactone form compound upon oral administration to a patient.

One class of oral formulations of the present invention comprises an amphiphilic block copolymer and a camptothecin. In these compositions the block copolymer increases intestinal absorption of the lactone form of a camptothecin due to its inhibitory effect with respect to BCRP, and other transporters, as well as metabolic enzymes. At the same time, the enhancing effect of the block copolymer on the carboxilate form of a camptothecin is less pronounced or is not seen at all. As a result, the AUC of the drug lactone form and lactone-to-carboxylate AUC ratio are significantly increased compared to those observed when the non-formulated drug was administered orally. Consequentially, upon oral administration to a patient, the oral bioavailability of the lactone form is increased significantly more than that of a directly related carboxylate form of the camptothecin or analog thereof.

With regard to each of the formulations described herein, the camptothecin compound preferably contains a lactone ring. According to these various embodiments, the pharmaceutical composition preferably has a pH less than 7, preferably a pH less than 6 and in one embodiment, a pH between 5 and 6.

Amphiphilic Block Copolymer

According to various embodiments, an amphiphilic block copolymer that provides an increased intestinal absorption of the lactone form of camptothecins is selected, without limitation, from the group of poly-ethylene oxide, polypropylene oxide; poly-ethylene oxide-alpha tocopherol; poly-ethylene oxide poly-alkyl; poly-ethylene oxide poly-lactide, poly(acrylic acid)-g-poly(ethylene oxide)-b-poly(propylene oxide)-b-poly(ethylene oxide) and the like, as well as other similar block or graft copolymers. Preferred compositions include those of poly-ethylene oxide and polypropylene oxide group, wherein the polyt(oxypropylene) portion of said block copolymer comprises at least 50% by weight of the block copolymer.

Pharmaceutical compositions are provided which comprise a closed alpha-hydroxy lactone form of camptothecin, or a closed alpha-hydroxy lactone form analog thereof that is effective for controlling abnormal cell proliferation, an amphiphilic block copolymer that increases the solubility of the lactone form compound under physiological conditions and selectively increases bioavailability of the lactone form compound, and at least one bioadhesive polymer that protects the lactone structure of the closed alpha-hydroxy lactone form of camptothecin or analog thereof and provides a sustained, controlled or delayed release of the drug in GI, which allows to improve its plasma pharmacokinetics by achieving a higher drug circulation time.

Pluronic

Polymeric surfactants such as the PEO-PPO-PEO block copolymers known under the generic name Poloxamers (polyether) and the trade names PLURONIC® (BASF Corporation, North Mount Olive, N.J.)), or Synperonic® (ICI), are employed as elements in aspects of many of the embodiments of the compositions of the present invention. Examples of these entities are illustrated in Table 1 as follows:

TABLE 1

| PLURONIC ® | Hydrophobe | Hydrophobe |
|---|---|---|
| (BASF) | Weight | Percentage |
| L31 | 950 | 90% |
| F35 | 950 | 50% |
| L42 | 1200 | 80% |
| L43 | 1200 | 70% |
| L44 | 1200 | 60% |
| L61 | 1750 | 90% |
| L62 | 1750 | 80% |
| L63 | 1750 | 70% |
| L64 | 1750 | 60% |
| P65 | 1750 | 50% |
| F68 | 1750 | 20% |
| P75 | 2050 | 50% |
| L81 | 2250 | 90% |
| P84 | 2250 | 60% |
| P85 | 2250 | 50% |
| F87 | 2250 | 30% |
| F88 | 2250 | 20% |
| L92 | 2750 | 80% |
| F98 | 2750 | 20% |
| L101 | 3250 | 90% |
| P103 | 3250 | 70% |
| P104 | 3250 | 60% |
| P105 | 3250 | 50% |
| F108 | 3250 | 20% |
| L121 | 4000 | 90% |
| L122 | 4000 | 80% |
| L123 | 4000 | 70% |
| F127 | 4000 | 30% |
| 10R5 | 1000 | 50% |
| 10R8 | 1000 | 20% |
| 12R3 | 1200 | 70% |
| 17R2 | 1700 | 80% |
| 17R1 | 1700 | 90% |
| 17R2 | 1700 | 80% |
| 17R4 | 1700 | 60% |
| 17R8 | 1700 | 20% |
| 22R4 | 2200 | 60% |
| 25R1 | 2500 | 90% |
| 25R2 | 2500 | 80% |
| 25R4 | 2500 | 60% |
| 25R5 | 2500 | 50% |
| 25R8 | 2500 | 50% |
| 31R1 | 3100 | 90% |
| 31R2 | 3100 | 80% |
| 31R4 | 3100 | 60% |

Bioadhesive Polymer

According to various compositions described herein the bioadhesive polymer component that provides an increased mucosal adhesion of the formulation in GI is a significant factor in the overall efficacy of the formulation. A key requirement is that the polymer must produce a bioadhesive interaction (“mucoadhesion”), described infra, when applied to the mucosal surface of intestine. The forces described herein refer to measurements made upon rat intestinal mucosa, unless otherwise stated.

Mucoadhesive or bioadhesive polymers (bioadhesive) can enhance local delivery of drugs by adhering to mucosal surfaces present on buccal, gastric, enteric, nasal, ophthalmic, pulmonary, or genitourinary tissues. Bioadhesive Polymers as elements of the compositions of the present invention include soluble and insoluble, biodegradable and nonbiodegradable polymers. Bioadhesive polymers in the compositions described herein can be hydrogels or thermoplastics, homopolymers, copolymers or blends, natural or synthetic. Preferred polymers include synthetic polymers having controlled swelling and adhesive properties. Most preferred polymers are copolymers of polyacrylic acid (PAA) and polyether, for example, which have unusually good bioadhesive properties when administered to the gastrointestinal tract. A requirement is that the polymer produces a bioadhesive interaction when applied to the mucosal surface of intestine.

In vitro and in vivo experiments also indicated that Pluronic-PAA copolymers [L. E. Bromberg and E. S. Ron, Adv. Drug Delivery Revs. 31, 197 (1998), L. Bromberg, J. Pharm. Pharmacol., 53, 109 (2001)] are mucoadhesive, to an extent at least equivalent or higher than PAA. Thus Pluronic-PAA copolymers, which is a preferred embodiment incorporated herein for a bioadhesive component of the invention, can effectively lengthen the residence time of the formulations on mucous surfaces and enhance bioavailability of the delivered drugs.

Two classes of polymers have appeared to show useful bioadhesive properties: hydrophilic polymers and hydrogels. In the large class of hydrophilic polymers, those containing carboxylic groups (e.g., poly[acrylic acid]) exhibit the best bioadhesive properties. In other studies, the most promising polymers were sodium alginate, carboxymethylcellulose, hydroxymethylcellulose and methylcellulose. Some of these materials are water-soluble, while others are hydrogels.

The graft-copolymers of PAA and Pluronic, whereby PAA and the polyether are linked via the C—C bond serve as a preferred embodiment herein. Aqueous solutions of the Pluronic-PAA copolymers exhibit no macroscopic phase separation, despite the abundance of the micellar aggregates formed above critical micellization concentration and temperature. Virtually any Pluronic can be utilized in the copolymer.

Rapidly bioerodible polymers such as poly[lactide-co-glycolide], polyanhydrides, and polyorthoesters, whose carboxylic groups are exposed on the external surface as their smooth surface erodes, are also suitable for bioadhesive drug delivery systems. In addition, polymers containing labile bonds, such as polyanhydrides and polyesters, are well known for their hydrolytic reactivity.

Representative synthetic polymers include polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes and copolymers thereof. Other polymers of interest include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly(ethylene terephthalate), poly(vinyl acetate), polyvinyl chloride, polystyrene, polyvinyl pyrrolidone, and polyvinylphenol. Representative bioerodible polymers include polylactides, polyglycolides and copolymers thereof, poly(ethylene terephthalate), poly(butic acid), poly(valeric acid), poly(lactide-co-caprolactone), poly[lactide-co-glycolide], polyanhydrides, polyorthoesters, blends and copolymers thereof.

Negatively charged hydrogels, such as alginate and carboxymethylcellulose, with exposed carboxylic groups on the surface, are appropriate bioadhesive systems, as well as some positively-charged hydrogels, such as chitosan.

Methods

Compositions of the present invention are intended for the prevention and/or treatment of conditions related to abnormal cell proliferation such as cancer, as well as hematological malignancy and tumors. Accordingly, compositions of the present invention are advantageously administered to a patient in need of treatment or otherwise suffering from a condition related to abnormal cell proliferation. Patient, as used herein, refers to a mammalian subject, including, for example, a human. In one embodiment, the method comprises: providing a pharmaceutical composition according to the present invention; and administering a therapeutically effective amount of the pharmaceutical composition orally to a patient in need thereof.

Embodiments

In one embodiment, a pharmaceutical composition of the present invention comprises at least one amphiphilic block copolymer such as Pluronic P85 and a water-soluble derivative of camptothecin such as topotecan. In this embodiment Pluronic P85 or another similar block copolymer does not change the chemical stability of the drug (as illustrated in respective following examples). However, it does selectively increase intestinal absorption of the lactone form of the drug leading to increased plasma AUC of the lactone and increased lactone-to-carboxylate ratio.

In another embodiment, a pharmaceutical composition comprises at least one amphiphilic block copolymer such as Pluronic P85 and a poorly-soluble camptothecin such as 9-nitro-20(S)-camptothecin. Upon dissolution the drug is solubilized into the block copolymer micelles and is preferably located in the water non-miscible hydrophobic core of the micelles. In this embodiment in addition to the selective increase in intestinal absorption of the drug lactone form, the block copolymer micelles further provide an increased solubility of the drug and increased stability of the lactone by isolating it from the aqueous environment, which reduces the rate of the lactone hydrolysis.

In yet another embodiment, a pharmaceutical composition is provided which comprises at least one amphiphilic block copolymer such as Pluronic P85, a camptothecin or its derivative and a component that increases mucosal adhesion of the composition such as polyacrylic acid that is either admixed to the composition or conjugated to the amphiphilic block copolymer. In this embodiment, in addition to selective increase in lactone intestinal absorption and increased solubility of the drug, the composition further provides sustained release properties that allow for an additional control of the drug plasma pharmacokinetics.

In a further preferred embodiment, a poly(acrylic acid)-g-polyether composition is provided that is mucoadhesive, impedes the hydrolysis of the camptothecin drug (irinotecan), and enhances the bioavailability of the drug by sustained release.

Mucoadhesion

Bioadhesion of polymers, i.e. the ability of macromolecules to adhere to biological tissues, is a phenomenon that refers to the formation of any bond between a biological and synthetic surface [D. E. Chickering and E. Mathiowitz, in Bioadhesive Drug Delivery Systems. Fundamentals, Novel Approaches, and Development, edited by E. Mathiowitz, D. E. Chickering, and C.-M. Lehr, Marcel Dekker, New York (1999), pp. 1-23]. If the biological substrate is a mucous membrane, the bioadhesion occurs between the mucus layer and a polymer and this phenomenon is called mucoadhesion. Since mucous membranes are the tissues which topical drug delivery formulations (gastrointestinal, buccal, ocular, nasal, oral, rectal, and vaginal) are administered to be in contact with, the mucoadhesion is useful in prolonging and controlling drug delivery [S. Tamburic and D. Q. M. Craig, in Chemical Aspects of Drug Delivery Systems, edited by D. R. Karsa, R. A. Stephenson, The Royal Society of Chemistry, Cambridge, UK (1996) pp. 11-40].

Studies refer to wetting of the mucus by the polymer followed by interpenetration and entanglement of the polymer and mucus components and polymer-mucin bond formation as the primary processes affecting mucoadhesion to various degrees [D. E. Chickering and E. Mathiowitz, in Bioadhesive Drug Delivery Systems. Fundamentals, Novel Approaches, and Development, edited by E. Mathiowitz, D. E. Chickering, and C.-M. Lehr, Marcel Dekker, New York (1999), pp. 1-23; D. Duchene, F. Touchard, and N. A. Peppas, Drug Dev. Ind. Pharm., 14, 283 (1988)]. It is stated [N. A. Peppas and P. A. Buri, J. Control. Release, 2, 257 (1985)] that to be mucoadhesive, a polymer solution should possess at least one of the following properties: ability to form hydrogen bonds, anionic charge, high molecular weight and sufficient flexibility to penetrate the mucus network, and surface tension that induces spreading over the mucus. Many mucoadhesive polymers are polyanions with carboxyl groups [S.-H. S. Leung and J. R. Robinson, In Polyelectrolyte Gels. Properties, Preparation, and Applications, Ed. by R. S. Harland, R. K. Prud'homme, American Chemical Society, Washington, D.C. (1992) ACS Symposium Series 480, pp. 269-284; H. S. Leung, J. R. Robinson, in Water-Soluble Polymers. Synthesis, Solution Properties, and Applications, edited by S. W. Shalaby, C. L. McCormick, and G. B. Butler, American Chemical Society, Washington, D.C. (1991) ACS Symposium Series 467, pp. 350-366]. However, a variety of nonionic (cellulose ethers, poly(vinyl alcohol), poly(ethylene oxide), etc.) and cationic (chitosan) mucoadhesive polymers are known [S. Tamburic and D. Q. M. Craig, in Chemical Aspects of Drug Delivery Systems, edited by D. R. Karsa, R. A. Stephenson, The Royal Society of Chemistry, Cambridge, UK (1996) pp. 11-40]. In addition to the electrostatic interactions, wetting and spreading are recognized as important factors in bioadhesion, contribution of hydrophobic interactions is also important. Despite some body of literature [J. M. Gu, J. R. Robinson, and S. H. Leung, Crit. Rev. Ther. Drug Carrier Syst., 5, 21 (1988); P. O. Glantz, T. Arnebrant, T. Nylander, and R. E. Baier, Acta Odontol. Scand., 57, 238 (1999); R. Bos, H. C. Van der Mei, and H. J. Busscher, FEMS Microbiol. Rev., 23, 179 (1999)], a universally accepted definition of the role of polymer hydrophobicity in bioadhesion is absent. This can be attributed to deficiencies in mechanistic understanding of the organization and physicochemical properties of the mucin layer.

The backbone of mucins is a protein that is densely substituted with oligosaccharides, primarily via o-glycoside linkages to the serine and threonine residues. The length of the hydrophilic side-chain oligosaccharides varies from one to twenty sugar residues. Several glycoprotein chains (subunits) are often linked to each other by disulfide bonds, comprising large (Mᵣ 10-40 million Da) structures [C. Wickström, J. R. Davies, G. V. Eriksen, E. C. Veerman, and I. Carlstedt, Biochem. J., 334, 685 (1998); D. J. Thornton, J. R. Davies, M. Kraayenbrink, P. S. Richardson, J. K. Sheehanand I. Carlstedt, Biochem. J., 265, 179 (1990); I. Carlstedt and J. K. Sheehan, Biorheology, 21, 225 (1984)]. Within the mucin apoprotein backbone, hydrophilic domains with a very high density of oligosaccharide grafting alternate with naked domains devoid of saccharide residues [I. Carlstedt and J. K. Sheehan, Biorheology, 21, 225 (1984); J. K. Sheehan, D. J. Thornton, M. Somerville, and I. Carlstedt, Am. Rev. Respir. Dis., 144, S4 (1991)]. These naked protein segments can fold and unfold in response to changes in ionic strength, are susceptible to proteolytic attack, and are at least partly responsible for the interactions of mucins with enzymes, inorganic cations, fatty acids, albumins, and bacteria [L. E. Bromberg, D. P. Barr, Biomacromolecules, 1, 325 (2000)].

Some models of the structure of bovine cervical mucin assume cross-linking via disulfide bridges and interactions among hydrophobic segments [K. S. P. Bhushana-Rao and P. L. Masson, in Mucus in Health and Disease, edited by M. Elstein and D. V. Paske, Plenum Press, New York (1977), pp. 275-282]. The sol-gel transition of gastric mucin at low pH is due to cross-linking of the mucin macromolecules through hydrophobic interactions [X. Cao, R. Bansil, K. R. Bhaskar, S. T. Bradley, J. T. LaMont, N. Niu, and N. H. Afdhal, Biophys. J. 76, 1250 (1999)]. At low pH, the mucin macromolecules adopt extended conformation that would expose the hydrophobic segments of the mucin molecules thus making them available for interchain aggregation. The aggregates would act as cross-links.

In vitro and in vivo experiments indicated that Pluronic-PAA copolymers [L. E. Bromberg and E. S. Ron, Adv. Drug Delivery Revs. 31, 197 (1998), L. Bromberg, J. Pharm. Pharmacol., 53, 109 (2001)] are mucoadhesive, to an extent at least equivalent or higher than PAA. Thus Pluronic-PAA copolymers, which is a preferred embodiment incorporated herein for a bioadhesive component of the invention, can effectively lengthen the residence time of the formulations on mucous surfaces and enhance bioavailability of the delivered drugs.

As discussed above, mucoadhesive polymers can enhance local delivery of drugs by adhering to mucosal surfaces present on buccal, gastric, enteric, nasal, ophthalmic, pulmonary, and genitourinary tissues. Mucoadhesive unmodified polyelectrolytes are typically ionized at physiological pH and thus would rapidly swell and dissolve in contact with biological fluids, eroding from the site of administration. The ability of polyelectrolytes to enhance the residence time of the drug associated with such polymers would then be compromised. A variety of formulation approaches have been developed aiming at the enhancement of the drug residence time and to lowering the release rate, while maintaining the mucoadhesive properties of the polyelectrolyte. Typically, a polyelectrolyte is mixed with a more hydrophobic polymer to result in a blend with enhanced drug-polymer interactions and higher viscosity. Since administration of highly viscous formulations is cumbersome, it is often preferred that a liquid drug-polymer formulation would gel at the site of administration. Such in situ gelling systems undergo reversible sol-gel transitions in response to temperature, pH, or ion composition of the fluids [A. Joshi, S. Ding, K. J. Himmelstein, U.S. Pat. No. 5,252,318, Oct. 12, 1993; K. J. Himmelstein and C. L. Baustian, U.S. Pat. No. 5,599,534, Feb. 4, 1997; S. Kumar and K. J. Himmelstein, J. Pharm. Sci., 84, 344 (1995) 93. S. Kumar, B. O. Haglund, and K. J. Himmelstein, J. Ocul. Pharmacol., 10, 47 (1994); A. Rozier, C. Mazuel, J. Grove, and B. Plazonnet, Int. J. Pharm., 57, 163 (1989); S. C. Miller, M. D. Donovan, Int. J. Pharm., 12, 147 (1982); J. L. Haslam, T. Higuchi, and A. R. Mlodozeniec, U.S. Pat. No. 4,474,752 (1984); K. Lindell and S. Engström, Int. J. Pharm., 95, 219 (1993); H.-R. Lin, K. C. Sung, J. Controlled Release, 69, 379 (2000); 0. Felt, V. Baeyens, M. Zignani, P. Buri, and R. Gumy, In Encyclopedia of Controlled Drug Delivery, edited by E. Mathiowitz, Wiley, New York (1999), Vol. 2, pp. 605-629]. However, the formulations based on physical blends are colloidally unstable and tend to either phase separate or dissociate at physiological pH. Stability of the in situ gelling systems can be addressed by utilization of benign polyelectrolyte block-copolymers. Surfactant-modified poly(acrylic acid) (PAA) is an in situ gelling system of choice [L. Bromberg, in Handbook of Surfaces and Interfaces of Materials, edited by H. S. Nalwa, Academic Press, New York (2001), Vol. 4, Chapter 7; L. Bromberg and E. Magner, Langmuir, 15, 6792 (1999); L. Bromberg and M. Temchenko, Langmuir, 15, 8627 (1999); L. Bromberg, J. Phys. Chem. B 102, 10741 (1998); L. Bromberg, Langmuir, 14, 5806 (1998); A. K. Ho, L. E. Bromberg, A. J. O'Connor, J. M. Perera, G. W. Stevens, and T. A. Hatton, Langmuir, 17, 3538 (2001); L. E. Bromberg and D. P. Barr, Macromolecules, 32, 3649 (1999); L. E. Bromberg, J. Pharm. Pharmacol., 53, 541 (2001); L. Bromberg, J. Pharm. Pharmacol., 53, 109 (2001)].

PAA is the industry benchmark for mucoadhesive polymers [H. Park and J. R. Robinson, Pharm. Res., 4, 457 (1987); S. Anlar, Y. Capan, and A. A. Hincal, Pharmazie, 48, 285 (1993); S. Tamburic and D. Q. Craig, Pharm. Res., 13, 279 (1996)]. The PAA bonding with benign polymeric surfactants such as the PEO-PPO-PEO block copolymers known under the generic name Poloxamers and the trade names Pluronic® (BASF) or Synperonic (ICI), results in a thermo- and pH-sensitive, in situ gelling system that has stability absent in a physical blend. Pluronics are the only up-to-date synthetic thermoviscosifying polymers approved by the U.S. Food and Drug Administration as food additives and pharmaceutical ingredients [BASF Performance Chemicals. FDA and EPA status. BASF Corporation, North Mount Olive, N.J., 1993]. Combination of PAA, generally recognized as safe in topical formulations [Carbopol®. The Proven Polymers in Pharmaceuticals. Bulletin 14. BF Goodrich Specialty Chemicals, Cleveland, Ohio, 1994] and Pluronic, although resulting in a novel polymer, is benign and has a better regulatory status compared to most other pH- and temperature-sensitive polymers. In vitro release studies showed that the gelation of the drug-polymer solution at 34-37° C. leads to a significantly prolonged drug release rate as well as to lower erosion (dissolution) rate of the polymers and drugs from the gels. The graft-copolymers of PAA and Pluronic, whereby PAA and the polyether are linked via the C—C bond [L. E. Bromberg, T. H. E. Mendum, M. Orkisz, E. S. Ron, and E. C. Lupton, Proc. Polym. Mater. Sci. Eng., 76, 273 (1997); M. J. Orkisz, L. Bromberg, R. Pike, E. C. Lupton, and E. S. Ron, Proc. Polym. Mater. Sci. Eng., 76, 276 (1997); L. Bromberg, M. Orkisz, E. Roos, E. S. Ron and M. Schiller, J. Control. Release, 48, 305 (1997); L. E. Bromberg, T. H. E. Mendum, M. J. Orkisz, E. C. Lupton and E. S. Ron, Polym. Prepr., 38, 602 (1997); L. E. Bromberg, M. J. Orkisz and E. S. Ron, Polym. Prepr., 38, 626 (1997); L. Bromberg, J. Phys. Chem. B, 102, 1956 (1998); L. E. Bromberg and M. G. Goldfeld, Polym. Prepr., 39, 681 (1998); L. Bromberg, Macromolecules, 31, 6148 (1998); L. Bromberg, Ind. Eng. Chem. Res., 37, 4267 (1998); L. Bromberg, J. Phys. Chem B, 102, 10736 (1998); L. Bromberg and L. Salvati, Bioconjugate Chem., 10, 678 (1999); P. D. T. Huibers, L. E. Bromberg, B. H. Robinson, and T. A. Hatton, Macromolecules, 32, 4889 (1999); L. E. Bromberg, M. Temchenko, and R. H. Colby, Langmuir, 16, 2609 (2000); L. E. Bromberg and D. P. Barr, Polym. Prepr., 41, 1709 (2000); N. Plucktaveesak, L. E. Bromberg, and R. H. Colby, Proc. XIIIth International Congress on Rheology, Cambridge, UK, 2000, Vol. 3, pp. 307-309; L. Bromberg, Ind. Eng. Chem. Res., 40, 2437 (2001); L. Olivieri, M. Seiller, L. Bromberg, E. Ron, P. Couvreur, and J.-L. Grossiord, Pharm. Res., 18, 689 (2001); L. Bromberg, E. C. Lupton, M. E. Schiller, M. J. Timm, G. W. McKinney, M. Orkisz, B. Hand, H. L. Emerson, and H. H. Doyle, Int. Patent Appl. WO 98/29487 (1998); E. S. Ron, L. Bromberg, M. Orkisz, M. Kearney, S. Luczak, M. J. Timm, S. J. Wrobel, Int. Patent Appl. WO 98/06438 (1998); E. S. Ron, B. J. Hand, L. Bromberg, M. Kearney, M. E. Schiller, P. M. Ahearn, S. Luczak, and T. H. E. Mendum, Int. Patent Appl. WO 98/50005 (1998); L. Bromberg, E. C. Lupton, M. E. Schiller, M. J. Timm, and G. McKinney, U.S. Pat. No. 5,939,485 (1999); E. S. Ron, L. E. Bromberg, M. Temchenko, Int. Patent Appl. WO007603A2 (2000)] serve as a preferred embodiment herein. Aqueous solutions of the Pluronic-PAA copolymers exhibit no macroscopic phase separation, despite the abundance of the micellar aggregates formed above critical micellization concentration and temperature [L. Bromberg, Ind. Eng. Chem. Res., 37, 4267 (1998); P. D. T. Huibers, L. E. Bromberg, B. H. Robinson, and T. A. Hatton, Macromolecules, 32, 4889 (1999)]. Aggregation in Pluronic-PAA solutions is unrelated to the LCST of the parent Pluronic, so that virtually any Pluronic can be utilized in the copolymer

### EXAMPLES

### Example 1

Analysis of Partitioning of Topotecan in Formulation with Pluronic F127

Partitioning coefficient P describes the tendency of molecules of topotecan to stay inside micelles formed by the carrier: P=[x]ₘ/[x]_{w}, where [x]ₘ is the actual concentration of topotecan inside micelles, [x]_{w} is the actual concentration of topotecan in aqueous phase. The carrier investigated in this example is pluronic F127 (BASF). Partitioning coefficient was estimated using the fluorescence dependence on the concentration of the carrier (Kabanov A. et al. (1995), Macromolecules 28, 2303-2314), by fitting the measured fluorescence of topotecan to the equation:

(Iₘₐₓ−I₀)/(−I₀)−1=1/(θP)−1/P

where:

I—fluorescence

Iₘₐₓ—fluorescence at maximum concentration of carrier

I₀—fluorescence at minimum concentration of carrier

P—partitioning constant

θ=0.01v([carrier]−CMC_{carrier}), volume portion of the micellar phase

CMC_{carrier}=0.02%—critical micelle concentration for Pluronic 127

v—partial specific volume of micelles, approximately v=0.8

[carrier]—concentration of the carrier.

30 microliter of 0.1 mM (0.046 mg/mL) solution of topotecan hydrochloride in methanol was deposited in each well of 96 well quartz plate (Hellma), and the solvent was allowed to evaporate under nitrogen. The plate was dried in vacuum. Solutions of the carrier were applied to wells, 300 microliter to each well. The concentration of carrier in solutions varied from 0.003% to 10%. The final concentration of topotecan was 0.010 mM. The plate was incubated 2 hrs at 37° C., and the fluorescence of the solution (excitation 360 nm, emission 530 nm) was measured using plate reader FL600 (BioTek). The partitioning coefficient estimated based on the dependence of topotecan fluorescence on carrier concentration was about 1 indicating that there was no preferred accumulation of topotecan in the polymer micelles.

### Example 2

Analysis of Solubility of Topotecan in Formulations with Pluronic P85

Calibration. Fluorescence of 0.300 mL samples of topotecan solutions in methanol containing between 0.00006 and 0.001 mg/mL, in triplicates on Helma Quarz plate, was measured using Bio-Tek FL600 fluorescence plate reader with excitation filter wavelength/bandwidth 360/40 nm and emission filter 530/25 nm, sensitivity 70. The average fluorescence was fitted to a linear equation and gave:

[fluorescence]=2697697.11 C+61.22

where C=topotecan concentration [mg/mL].

Solubility. 1 mg topotecan samples, in triplicates, and 1 mL of the respective buffer or carrier solution, were incubated at room temperature with shaking for 1 hour. The samples were then filtered through Acrodisc CR, PTFE Syringe filter, 4 mm diameter, 0.45 μm pore size Gelman part No. 4472. The filtrate was diluted 100 times with methanol, and the final concentration of topotecan was determined by fluorescence measurement using the above-described calibration.

| Solution | Topotecan concentration [mg/mL] |
|---|---|
| PBS | 0.062 ± 0.005 |
| P85 1% in PBS | 0.095 ± 0.005 |
| P85 3% in PBS | 0.105 ± 0.005 |

Where PBS—phosphate buffered saline, pH 7.2

### Example 3

Analysis of Solubility of Camptothecin in Formulations with Pluronic P85

Calibration. Fluorescence of 0.300 mL samples of camptothecin solutions in methanol containing between 0.00003 and 0.0005 mg/mL, in triplicates on Helma Quarz plate, was measured using Bio-Tek FL600 fluorescence plate reader with excitation filter wavelength/bandwidth 360/40 nm and emission filter 530/25 nm, sensitivity 70. The average fluorescence was fitted to a linear equation and gave:

[fluorescence]=1493438.28 C+28.75

where C—camptothecin concentration [mg/mL].

Solubility. 0.1 mg camptothecin samples, in triplicates, and 1 mL of the respective buffer or carrier solution, were incubated at room temperature with shaking for 1 hour. The samples were then filtered through Acrodisc CR, PTFE Syringe filter, 4 mm diameter, 0.45 μm pore size Gelman part No. 4472. The filtrate was diluted 100 times with methanol, and the final concentration of camptothecin was determined by fluorescence measurement using the above-described calibration.

| Solution | Camptothecin concentration [mg/mL] |
|---|---|
| PBS | 0.0000 ± 0.0015 |
| P85 1% in PBS | 0.0031 ± 0.0015 |
| P85 3% in PBS | 0.0049 ± 0.0015 |

Where PBS—phosphate buffered saline, pH 7.2

### Example 4

Synthesis of Pluronic-PAA Microgel

Nonionic copolymers Pluronic F127 NF and L92 were obtained from BASF Corp. and used without further treatment. Acrylic acid (99%, vinyl monomer), ethylene glycol dimethacrylate (98%, divinyl cross-linker), dodecane (99+%, solvent), and 4,4′-azobis(4-cyanovaleric acid) (75+%, azo initiator) were purchased from Aldrich Chemical Co. and used as received. Lauroyl peroxide (97%, redox initiator) was obtained from Fluka Chemie AG (Switzerland). Poly(vinylpyrrolidinone-co-1-hexadecene) (Ganex V-216) (dispersion stabilizer) was obtained from International Specialty Products (Wayne, N.J.). Doxorubicin hydrochloride and taxol, both of 99% purity, were obtained from Hande Tech USA (Houston, Tex.), a subsidiary of Yunnan Hande Technological Development Co. (Kunming, P. R. China). Camptothecin and β-estradiol were obtained from Sigma-Aldrich Co. and used as received.

Gel synthesis was carried out on a laboratory scale in an adiabatic mode. Acrylic acid (40 mL) was partially neutralized by addition of 5 M NaOH aqueous solution (0.5 mL). Pluronic (24 g) was dissolved in the resulting solution under nitrogen and ethylene glycol dimethacrylate (EGDMA) (1.1 mL) was added. Lauroyl peroxide (100 mg) and 4,4′-azobis(4-cyanovaleric acid) (100 mg) were dissolved in 2 mL of acrylic acid and added to the solution of Pluronic in acrylic acid. The resulting solution was deaerated by nitrogen bubbling for 0.5 h and added to a 3-necked 0.5-mL flask containing 1 wt % solution of Ganex V-216 in dodecane (200 mL). The flask was vigorously stirred by a mechanical stirrer and deaerated by constant nitrogen purge from the bottom. Then the flask was heated to 70° C. using an oil bath and kept at that temperature under stirring and nitrogen purge. After about 1 h, formation of white particles was observed on the flask walls. The reaction was continued at 70° C. for another 3 h. Then the reactor was disassembled, and the contents of the reactor were filtered using Whatman filter paper (retention size 10 μm). The microgel particles were extensively washed by hexane and dried under vacuum. The level of the monomer in the wash-outs in such a procedure is typically only 1-2% of the initial acrylic acid loading, due to extremely high efficiency of the monomer incorporation into the copolymers. Spherical particles were observed under microscope. Particle sizing was performed in hexane using a ZetaPlus Zeta Potential Analyzer (Brookhaven Instruments Co.). A typical batch of particles with XL=1 mol % was measured to have effective median diameter of 13 μm and polydispersity of 1.4.

### Example 5

Loading of Camptothecin and Other Hydrophobic Drugs into Pluronic-PAA Microgel

The loading of the hydrophobic solutes such as taxol, camptothecin, and estradiol into microgels was measured by equilibrating solute adsorbed onto steel beads with the 1 wt % suspension of microgels (pH 7.0). Stainless steel beads (1-3 μm diameter) were soaked in 10 mM solution of the solute in either acetonitrile (taxol), dimethylsulfoxide (camptothecin), or absolute ethanol (estradiol, progesterone), following by stripping off the solvent under vacuum. The beads were used in order to enhance the area of contact between the microgel suspension and drug. The beads were separated into several fractions. One fraction was added to a polypropylene vial containing the microgel suspension (0.5 mL) and the vial was gently shaken in a horizontal position in an environmental chamber at 20 or 37° C. Then the beads were recovered from the suspension by using a magnet. The beads were dried under vacuum and placed into an appropriate solvent (0.5 mL), where the solute was extracted after shaking overnight. The solvent fraction was assayed for the solute concentration using HPLC. The control fraction of loaded beads was subjected to the extraction without equilibration with the microgel suspension. The solute concentrations were measured in triplicate using the HPLC system described above. The chromatography assay for taxol comprised the use of a Capcell Pak C18 UG 120 (150×4.6 mm I.D., particle size 3 μm) column (Phenomenex), acetonitrile-0.1% phosphoric acid in DI water (55:45 v/v, 1.3 mL/min) as a mobile phase, and UV detection at 227 nm. The typical retention time of the taxol peak was 3.46 min. For the camptothecin assay, a solution of the drug in DMSO (50 μL) was injected onto the aforementioned C₁₈ column and eluted at 1 mL/min flow rate and 40° C. using 0.1 M ammonium acetate (pH 5.6) and acetonitrile as mobile phase. A linear gradient of 45% to 85% acetonitrile was applied, and the UV/vis detection was carried out at 254 and 370 nm. The retention time of the camptothecin peaks was 7.5-8.5 min. The peak area was integrated and used to calculate camptothecin concentration using standard calibration curves. The HPLC assay of estradiol solutions in ethanol was performed as described in [Bromberg, L., Temchenko, M. Langmuir, 1999, 15, 8627].

Results

Solubilizing capacity of the Pluronic-PAA gels for hydrophobic, water-insoluble drugs is shown in Table 2 below. The more hydrophobic gels based on Pluronic L92 possess a higher capacity for the hydrophobic drugs. Taxol and other hydrophobic drugs are solubilized by the hydrophobic PPO chains in the gels. The latter notion is supported by the fact that the equilibrium solubility of taxol and the other hydrophobic drugs under study in water is 16-50 less than the equilibrium solubility in 1 wt % aqueous gel suspension. The solubilizing capacity of the microgels for taxol and steroid hormones is at least equal to that of the Pluronic-PAA micelles [Bromberg, L., Temchenko, M. Langmuir, 1999, 15, 8627]. The characteristic increase in the drug loading capacity at higher temperatures (compare results at 20 and 37° C.) above the critical aggregation temperatures provides additional evidence for the mechanism of hydrophobic drug solubilization into micelle-like aggregates within the gels via entropic interactions. The micelles in Pluronic-PAA solutions typically have solubilizing capacity similar to that in the Pluronic aqueous solutions [Bromberg, L., Temchenko, M. Langmuir, 1999, 15, 8627]. The least hydrophobic of the four drugs, camptothecin, showed higher uptake by the microgels. This can be explained by the fact that the loading of the amphiphilic solute can occur both via the solubilization into hydrophobic PPO cores of the aggregates and entrapment into more hydrophilic core-swollen gel interface.

TABLE 2
Uptake of Camptothecin and Hydrophobic
Drugs by Pluronic-PAA Gels

| | | Log | Uptake ± S.D. (μmol/g gel) | |
|---|---|---|---|---|
| Drug | MW | P | F127-based gel | L92-based gel |
| Camptothecin | 348.4 | 0.87 | 16.3 ± 1.86 (37° C.) | 19.5 ± 2.33 (37° C.) |
| | | | 13.4 ± 1.54 (20° C.) | 16.2 ± 1.87 (20° C.) |
| Taxol | 853.3 | 4.0 | 6.97 ± 0.87 (37° C.) | 7.25 ± 0.74 (37° C.) |
| | | | 2.27 ± 0.90 (20° C.) | 3.65 ± 0.43 (20° C.) |
| β-Estradiol | 272.4 | 3.86 | 8.12 ± 0.39 (37° C.) | 9.68 ± 0.24 (37° C.) |
| | | | 4.55 ± 0.46 (20° C.) | 6.32 ± 0.64 (20° C.) |

### Example 6

Stability of Topotecan Lactone in Pluronic P85 Formulation

The formulation was prepared by mixing the stock solution containing 6 mg/mL of topotecan in 0.1% aqueous acetic acid with the 2.05% P85 solutions in PBS 1:20 (v/v), to obtain final compositions containing 0.3 mg/mL drug in 1% P85. The control formulation was prepared by simple dilution of topotecan stock solution with PBS 1:20 (v/v). The initial time of incubation (O hour) is the time of mixing of the respective stock solutions. The samples were incubated at 37° C. in thermostat. After various time intervals (1, 2, 3, 6, and 10 hrs) post-injection, a sample of formulation was taken, diluted 1000 times with the HPLC mobile phase, and injected into HPLC. The HPLC conditions were following:

C₁₈ reversed phase column 250×4.6 mm, Phenomenex C18 (2) Luna, 5 μm, column temperature 40° C., flow rate 1 mL/min, injection volume 20 μL, fluorescence detection at wavelengths λ_{excitation}=381 nm, λₑₘᵢₛₛᵢₒₙ=525 nm, mobile phase 94% of 2% triethylamine acetate pH 5.5/6% acetonitrile, run time 7 min

The results of topotecan lactone stability are represented as percentage of 0 hour in the Table 3:

| | | | | | 6 | 10 |
|---|---|---|---|---|---|---|
| Formulation | 0 hour | 1 hour | 2 hours | 3 hours | hours | hours |
| Control, [% of 0 h] | 100.0 | 76.0 | 56.4 | 49.7 | 45.9 | 44.8 |
| 1% P85, [% of 0 h] | 100.0 | 78.2 | 59.6 | 51.6 | 47.5 | 46.4 |

### Example 7

Enhancement of Irinotecan (CPT-11) Lactone Stability in Pluronic-PAA Microgel

Experiments were conducted to evaluate the enhancement of camptothecin derivative, irinotecan (CPT-11) (Pharmacia, Kalamazoo, Mich.) stability in human serum. It is well-known in the art [T. G. Burke, Z. Mi, Analyt. Biochem., 1993, 22, 285-287; T. G. Burke, A. K. Mishra, M. C. Wani, M. E. Wall, Biochemistry, 1993, 32, 5352-5364] that the lactone ring of camptothecin and its analogs is unstable when it binds to human serum albumin and some other proteins and enzymes present in human serum. Hence, in this Example we conducted stability tests using Pluronic-PAA gels.

Experimental

The gel samples were loaded by CPT-11 as described in Example 1 at 37° C. The loading of F127-PAA and L92-PAA gels was 19.6±3.2 and 23.0±2.8 μmol/g gel, respectively. After loading, the gels were snap-frozen in liquid nitrogen and lyophilized. The gel samples were stored at −70° C.

Liquid chromatography assays were conducted using a Hewlett-Packard Series 1100 HPLC system, which included an HP 1046A fluorescence detector, a thermostatted autosampler, pumps, and a column compartment with a Capcell Pak C18 UG 120 (150×4.6 mm I.D., particle size 3 μm) column. Mobile phase consisted of 75 mM ammonium acetate buffer (pH 6.4)-acetonitrile (78:22, v/v), to which tetrabutylammonium dihydrogen phosphate was added to result in 5 mM concentration. The column was eluted at a flow rate of 1.4 mL/min. The gel sample was equilibrated with excess human serum from clotted male whole blood (Sigma Chemical Co.) for specified time at 37° C. under constant shaking (200 rpm). Then the gel pre-loaded with CPT-11 was filtered from the serum using filter paper (Whatman, retention size 10 μm), rinsed on the filter by ice-cold water and lyophilized. For the irinotecan assay, 10 mg of dry gel were suspended in DMSO (100 μL) and extracted at ambient temperature under constant shaking. The gel was removed from the suspension by ultracentrifugation (15,000×g) for 15 min. The supernatant was filtered through a 0.22 μm membrane filter and 50 μL of the solution was then injected onto the HPLC column. Fluorescence detection was carried out at an excitation wavelength of 375 nm and emission wavelength of 420 nm. In the control experiments, a 1 mg/mL stock solution of CPT-11 in 10 mM HCl was prepared. The stock solution was diluted into human serum at a ratio of 1:75 v/v and the time commenced. The serum spiked with CPT-11 was kept at 37° C. under constant shaking, and 50 μL samples were withdrawn intermittently and immediately frozen to −70° C. The thawed samples were diluted 5-fold by chilled (−30° C.) methanol and centrifuged for 2 min at 10,000×g. The supernatant was analyzed by HPLC as described above. Irinotecan metabolites SN-38 and APC were obtained in pure form from Pharmacia (Kalamazoo, Mich.) and were used for peak identification.

Results

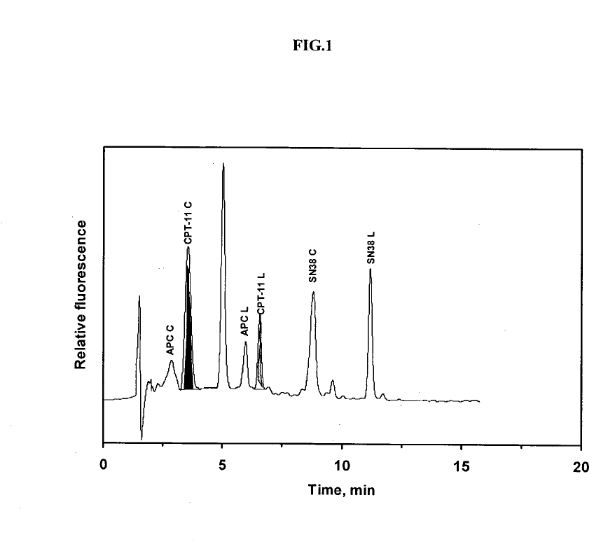

A chromatogram showing the metabolism of CPT-11 in human serum (15 min incubation) is shown in FIG. 1. As is seen, without encapsulation into gels a substantial portion of CPT-11 was metabolised.

FIG. 1 shows a representative chromatogram of CPT-11 and its metabolites APC and SN38 obtained after equilibration of CPT-11 in human serum for 10 min. Unidentified peaks may represent SN38 glucuronide and some other metabolites [E. Gupta, T. M. Lestingi, R. Mick, J. Ramirez, E. E. Vokes, M. J. Ratain, Cancer Res., 1994, 54, 3723]. The highlighted peaks were integrated to obtain total concentration of lactone and carboxylate forms of CPT-11.

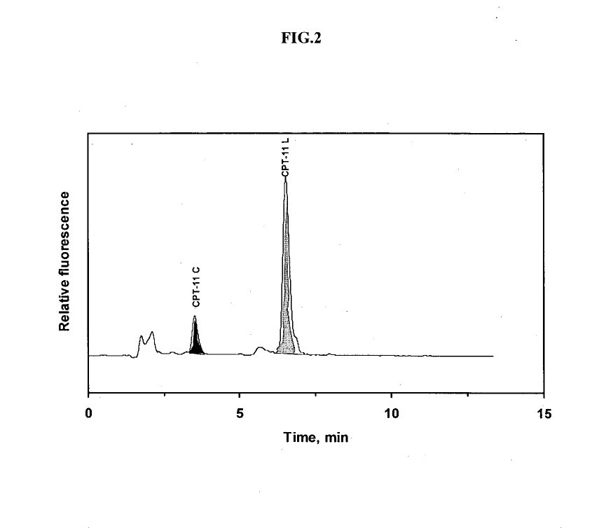

In marked contrast to the above observations, no metabolites of CPT-11 were discernible in chromatograms of the gels pre-loaded with CPT-11 and subsequently kept in human serum (FIG. 2), providing evidence that the CPT-11 was much less accessible to the enzymatic hydrolysis. Such useful retardation of the drug hydrolysis by enzymes via protective action of Pluronic-PAA has been observed with proteins as well [L. Bromberg, Interactions among proteins and hydrophobically modified polyelectrolytes, J. Pharm. Pharmacol., 53(4) (2001) 541-547].

We were tempted to estimate the extent of the lactone hydrolysis of CPT-11 with and without gel protection.

FIG. 2 shows a representative chromatogram of CPT-11 obtained after equilibration of gel based on Pluronic F127-PAA loaded with CPT-11 in human serum for 1 h.

The results of HPLC analysis are presented in Table 4 below:

TABLE 4
Results of HPLC analysis of CPT-11 hydrolytic
products obtained in human serum without gel
and with a gel based on Pluronic F127-PAA
loaded with CPT-11.

Relative Peak Area

| | | With Pluronic- |
|---|---|---|
| | Without Gel | PAA-EGDMA |
| Metabolite | (exposure 10 min) | gel (exposure 1 h) |
| CPT-11L | 1.00 | 4.54 |
| CPT-11C | 2.39 | 1.00 |
| APC-C | 1.73 | not detected |
| APC-L | 0.778 | not detected |
| SN28-c | 1.18 | not detected |
| SN28-L | 1.30 | not detected |

The above Table 4 shows dramatic stabilization of the lactone form of the drug by encapsulation in the Pluronic-PAA gel.

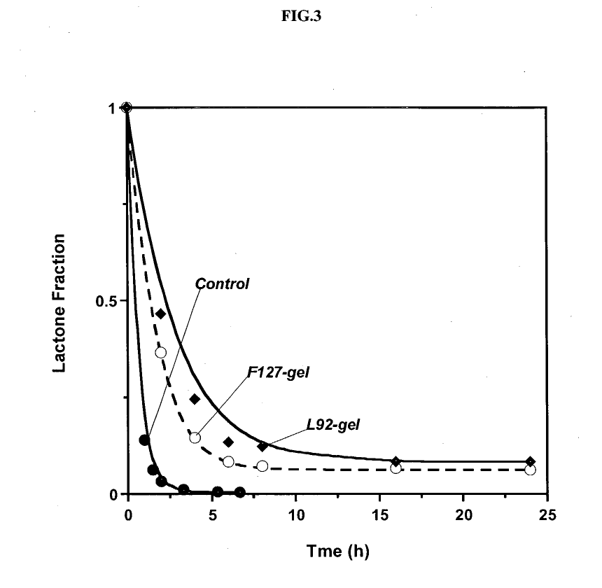

The effective half-life of the lactone hydrolysis was calculated from a first-order expression t_{1/2}=ln(2/k), where k is the rate constant. The fraction of the residual CPT-11 lactone in the medium versus time FL was fitted to the expression F_{L}=F_{∝}+F₀¹exp(−kt), where F_{∝} and F₀¹=F₀^{total}−F_{∝} correspond to the lactone fraction at equilibrium (t→∞) and at the commencement (t=0) of the kinetics, respectively. The results of the kinetic experiments are shown in FIG. 3. As is seen, CPT-11 unprotected by the gels was hydrolysed much more rapidly, while the gels proved to be effective barriers against the drug decomposition.

FIG. 3 shows kinetic evaluation of the rate of lactone ring opening for CPT-11 in human serum without gels (filled circles) and with L92-PAA gels (filled diamonds) or with F127-PAA gels (open circles).

TABLE 5
Kinetic evaluation of the rate of lactone ring opening
for CPT-11 in human serum without gels in serum
and with L92-PAA gels or with F127-PAA gels.

| | | | Relative | | Relative |
|---|---|---|---|---|---|
| | Relative | | lactone | | lactone |
| | lactone | | concentration | | concentration |
| | concentration | Time, | with F127- | Time, | with L92- |
| Time, h | in serum | h | PAA gel | h | PAA gel |
| 0 | 1.000 | 0 | 1.000 | 0 | 1.000 |
| 1 | 0.140 | 2 | 0.365 | 2 | 0.465 |
| 1.5 | 0.062 | 4 | 0.145 | 4 | 0.245 |
| 2 | 0.033 | 6 | 0.084 | 6 | 0.134 |
| 3.33 | 0.012 | 8 | 0.073 | 8 | 0.123 |
| 5.33 | 0.006 | 16 | 0.067 | 16 | 0.083 |
| 6.67 | 0.005 | 24 | 0.062 | 24 | 0.083 |

The parameter t_{1/2} was estimated to be 0.16, 1.1, and 1.7 h for CPT-11 without gels, with F127-PAA, and L92-PAA gels, respectively. Hence, the drug is about 10-fold more stable when encapsulated into gels.

### Example 8

This Example Illustrates Mucoadhesive Properties of the Gels Based on Pluronic-PAA Copolymers

Experimental

Experimental protocols involving animals were reviewed and approved by the animal experimentation committee at Supratek Pharma, Inc. Adult, pathogen-free male Sprague-Dawley rats (Harlan, 280-320 g) were acclimated to the environmentally controlled quarters (25+1° C. and 12:12 h light-dark cycle) for 5-6 days prior to the experiments. The animals had free access to the laboratory rodent diet and water, ad libitum, until 12 h prior to being used in experiments, at which time food only was withdrawn. At the start of experiments, the rats (n=4) were sacrificed by cervical dislocation after isofluorane anesthesia. The rat gastrointestinal tract was rapidly removed from the animal. Stomach, proximal and distal small intestine, and colon were quickly isolated and flushed with ice-cold physiological saline to remove the lumenal contents. The jejunum mucosa excised from the small intestine and all other tissues were snap-frozen in liquid nitrogen and stored at −70° C. Thus, frozen tissue utilized within 24 h of dissection was found to possess identical adhesive parameters to those observed with material used immediately after isolation from the animal. Analogous successful experiments on bioadhesion using rat intestine have been reported elsewhere [E. P. Kakoulides, J. D. Smart, J. Tsibouklis, Azocrosslinked poly(acrylic acid) for colonic delivery and adhesion specificity: in vitro degradation and preliminary ex vivo bioadhesion studies, J. Control. Rel., 1998, 54 (1) 95-109].

TA.XT2i Texture Analyzer (Texture Technologies Corp., Scarsdale, N.Y.) equipped with a 5-kg load cell was used in tensile strength measurements. The setup had a force measurement accuracy of 1 mN and a distance resolution of 1 μm. Two types of measurements were applied. In the first series of experiments, bioadhesion between the gels and mucosa was studied [H. Hägerström, K. Edsman, Interpretation of mucoadhesive properties of polymer gel preparations using a tensile strength method, J. Pharm. Pharmacol., 2001, 53, 1589-1599]. An equilibrium swollen gel sample (15 mL, swelling medium, physiological saline, 0.9% NaCl, pH 7.4; total polymer concentration, 1 wt %) was placed in a thermostatted cell with a 4.0 cm-circular opening and the cell was kept in refrigerator to ensure that air bubbles were not entrapped in the gel. The cell was mounted on the stationary surface of the instrument and the gel was brought to 37° C. A piece of the mucosa was thawed to room temperature and attached to the upper movable stainless steel cylindrical probe using Vetbond™ Tissue Adhesive (3M Company, St. Paul, Minn.). Care was taken to ensure that a flat surface of the ex vivo mucosa (total 1.54 cm² area) was exposed parallel to the stationary surface and the surface of the gel. The mucosa was lowered toward the gel surface at a speed of 1 cm/min. Upon contacting the gel surface, as detected by a triggering force of 2 mN, the mucosa penetrated into the gel with a speed of 0.1 mm/s to 1.0 mm depth. After 2.0 min contact time, the mucosa was withdrawn, again at the speed of 0.1 mm/s. Preliminary experiments where the withdrawal speed and contact time varied in 0.1-5 mm/s and 2-10 min ranges, respectively, indicated high precision and reproducibility of these measurements, as compared to higher withdrawal speeds and longer contact times. Cohesiveness of the mucus itself was measured by attaching mucosa to both the stationary surface and the probe. The penetration depth was only 0.5 mm, which ensured the complete contact between the two mucous surfaces. For mucus cohesiveness, 25 measurements were performed, and the mucosa used was obtained from at least 4 different rats. Literature data indicate that the above conditions are optimal in the hydrogel studies [H. Hägerström, K. Edsman, Interpretation of mucoadhesive properties of polymer gel preparations using a tensile strength method, J. Pharm. Pharmacol., 2001, 53, 1589-1599; C. M. Caramella, S. Rossi, M. C. Bonferoni, A Rheological Approach to Explain the Mucoadhesive Behavior of Polymer Hydrogels, in: Bioadhesive Drug Delivery Systems. Fundamentals, Novel Approaches, and Development, ed. By E. Mathiowitz, D. E. Chickering, C.-M. Lehr, Marcel Dekker, New York, 1999, pp. 25-65].

Results of the Mucoadhesion Study

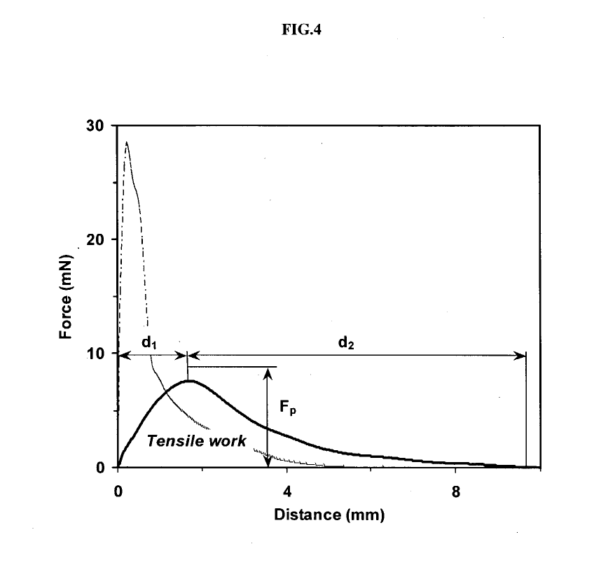

The tensile tests were interpreted as in FIG. 4 below.

FIG. 4 shows force-distance diagrams obtained for mucoadhesive F127-PAA gel preparations in contact with rat jejunum mucosa at 37° C. Total polymer concentration in isotonic saline (0.9% NaCl) was kept at 1 wt %. Solid line shows results with uncross-linked F127-PAA, while dashed line illustrates behavior of the cross-linked F127-PAA-EGDMA gel (cross-linking ratio EGDMA/AA 1 mol %). Indices Fₚ, d₁, and d₂ stand for the peak force, deformation to peak, and deformation peak to failure, respectively. The tensile work is estimated from the area under the curve, and total deformation to failure is a sum of d₁ and d₂.

The summary of results is collected in Table 6 below. As is seen, Pluronic-PAA gels were similar and in many instances exceeded mucoadhesive properties of Carbopol C934P, which is known in the art as an industry standard for mucoadhesive polymers.

TABLE 6
The Results of the Mucoadhesion Study of Pluronic-PAA
copolymers and Gels on Rat Intestine.^{a}

| | | Fracture | Total | |
|---|---|---|---|---|
| | Tensile work, | strength, | deformation to | Deformation to |
| Material^{b} | mJ | mN/cm² | failure, mm | peak, mm |
| L92-PAA | 0.025 ± 0.0015 | 4.82 ± 0.69 | 7.96 ± 2.14 | 1.69 ± 0.30 |
| L92-PAA-EGDMA | 0.028 ± 0.0031 | 18.5 ± 2.11 | 4.75 ± 1.27 | 0.22 ± 0.042 |
| F68-PAA | 0.019 ± 0.0018 | 4.18 ± 1.32 | 5.02 ± 1.51 | 1.98 ± 0.42 |
| F68-PAA-EGDMA | 0.030 ± 0.0032 | 21.2 ± 4.23 | 4.47 ± 1.63 | 0.20 ± 0.012 |
| F88-PAA | 0.023 ± 0.0024 | 4.07 ± 1.15 | 7.86 ± 1.36 | 1.97 ± 0.28 |
| F88-PAA-EGDMA | 0.020 ± 0.0044 | 17.1 ± 2.77 | 4.81 ± 1.32 | 0.19 ± 0.024 |
| F108-PAA | 0.032 ± 0.0026 | 4.69 ± 0.52 | 10.11 ± 1.55 | 1.80 ± 0.62 |
| F108-PAA-EGDMA | 0.019 ± 0.0031 | 16.5 ± 2.14 | 4.75 ± 1.02 | 0.17 ± 0.022 |
| P105-PAA | 0.016 ± 0.0031 | 4.01 ± 0.57 | 4.96 ± 1.43 | 1.79 ± 0.67 |
| P105-PAA-EGDMA | 0.023 ± 0.0055 | 18.2 ± 2.66 | 5.12 ± 1.32 | 0.21 ± 0.026 |
| P103-PAA | 0.018 ± 0.0022 | 4.11 ± 0.87 | 4.66 ± 1.25 | 0.85 ± 0.33 |
| P103-PAA-EGDMA | 0.021 ± 0.0043 | 18.56 ± 2.09 | 5.11 ± 1.56 | 0.21 ± 0.019 |
| L61-PAA | 0.032 ± 0.0014 | 5.28 ± 1.56 | 9.59 ± 2.32 | 1.23 ± 0.15 |
| L61-PAA-EGDMA | 0.023 ± 0.0043 | 14.8 ± 1.71 | 5.22 ± 1.11 | 0.23 ± 0.031 |
| F127-PAA | 0.035 ± 0.0023 | 5.62 ± 0.72 | 10.9 ± 3.12 | 2.08 ± 0.45 |
| F127-PAA-EGDMA | 0.047 ± 0.0067 | 22.5 ± 4.16 | 6.83 ± 2.21 | 0.31 ± 0.030 |
| F38-PAA | 0.0069 ± 0.0014 | 3.17 ± 0.64 | 3.91 ± 1.02 | 1.07 ± 0.56 |
| F38-PAA-EGDMA | 0.011 ± 0.0034 | 6.23 ± 1.28 | 5.15 ± 1.67 | 0.45 ± 0.036 |
| Carbopol C934P NF^{c} | 0.034 ± 0.0021 | 5.88 ± 2.03 | 5.26 ± 1.71 | 1.90 ± 0.39 |
| Carbopol C907 | 0.0061 ± 0.0011 | 3.13 ± 0.86 | 3.33 ± 1.10 | 0.86 ± 0.20 |
| Blend of Pluronic F127 | 0.0056 ± 0.0018 | 3.01 ± 0.62 | 3.26 ± 0.91 | 0.92 ± 0.31 |
| and PAANa (MW | | | | |
| 170000), 2.5 wt % each | | | | |
| Mucus | 0.028 ± 0.0010 | 19.3 ± 5.34 | 2.67 ± 1.02 | 0.20 ± 0.032 |

^{a}Only statistically significant data (P < 0.05) are reported. The mean values ±95% confidence intervals of the means (n = 6-25) are listed in the Table 6.
^{b}Letters and numbers stand for a particular Pluronic used to bind with poly(acrylic acid); EGDMA stands for ethylene glycol dimethacrylate cross-linker, used in gels at effective molar ratio of the monomer (acrylic acid) to cross-linker of 100:1.
^{c}Carbopol polymers were obtained from Noveon, Inc. (Cleveland, OH). Carbopol C934P is lightly cross-linked poly(acrylic acid), while Carbopol C907 is an uncross-linked poly(acrylic acid).

### Example 9

Plasma Pharmacokinetics of Topotecan upon i.v. Administration

Topotecan dosing solution was prepared as follows:

About 28 mg of topotecan were weighed and dissolved in 7 mL of isotonic solution. Topotecan was i.v. administered in normal rats at a dose of 15 mg/mL.

After various time intervals (15, 30 min, 1, 3, 6, and 10 hrs) post-injection, the blood samples were collected.

The blood samples were collected from the jugular vein with the tube heparinized and kept immediately in ice for 5 to 10 min. Blood was immediately centrifuged, and plasma was separated. The plasma samples were immediately frozen in dry ice and stored at −80° C. until further use.

The aliquots (100 μL) of plasma samples were extracted with 400 μL of cold methanol. The supernatant was separated by centrifugation at 10000 RPM for 10 minutes and kept at −80° C. before HPLC analysis. The HPLC conditions were the following:

C₁₈ reversed phase column 250×4.6 mm, Phenomenex C18 (2) Luna, 5 μm, column temperature 40° C., flow rate 1 mL/min, injection volume 20 μL, fluorescence detection at wavelengths λ_{excitation}=381 nm, λₑₘᵢₛₛᵢₒₙ=525 nm, mobile phase 94% of 2% triethylamine acetate pH 5.5/6% acetonitrile, run time 7 min

The topotecan lactone and topotecan carboxilate concentrations were calculated from the area under the peak (AUP) by using a calibration curve. The areas under the curves (AUC) were calculated by trapezoidal rule. The results are shown in the Table 7:

| | | | | AUC_{0-10 h} | Ratio |
|---|---|---|---|---|---|
| | Topotecan | Cₘₐₓ | Ratio Cₘₐₓ | [ng * | AUC_{0-10 h} |
| Formulation | form | [ng/mL] | Lac/Car | h/mL] | Lac/Car |
| Control | Lactone | 320.9 | 0.4 | 454.0 | 0.3 |
| | Caboxylate | 754.1 | | 1521.7 | |

### Example 10

Oral Bioavailability of Pluronic P85 Formulated and Unformulated Topotecan

Topotecan dosing solutions were prepared as follows:

About 15 mg of topotecan were weighed and dissolved in 5 mL of tap water for control, and about 15 mg of topotecan were dissolved in 5 mL of 1% P85 solution in tap water for formulation;

Both topotecan dosing solutions were orally administered by gavage in normal rats at a dose of 15 mg/kg. Three animals were used per each group.

After various time intervals (15, 30 min, 1, 3, 6, and 10 hrs) post-injection, the blood samples were collected.

The rats were anesthetized by general inhalation of Isoflurane (Bimeta-MTC, Animal Health Inc. Cambridge, ON, Canada).

The plasma sample collection, extraction and HPLC analysis were carried out with the same matter as is described in example 8.

The topotecan lactone and topotecan carboxylate concentrations were calculated from the area under the peak (AUP) by using a calibration curve. The areas under the curves (AUC) were calculated by trapezoidal rule. The results are shown in the Table 8:

| | | | | AUC_{0-10 h} | Ratio |
|---|---|---|---|---|---|
| | Topotecan | Cₘₐₓ | Ratio Cₘₐₓ | [ng * | AUC_{0-10 h} |
| Formulation | form | [ng/mL] | Lac/Car | h/mL] | Lac/Car |
| Control | Lactone | 20.3 | 1.2 | 80.5 | 1.2 |
| | Caboxylate | 16.2 | | 69.5 | |
| 1% P85 | Lactone | 67.4 | 2.8 | 171.1 | 1.7 |
| | Caboxylate | 23.9 | | 100.5 | |

### Example 11

Oral Administration of Topotecan Formulated with Pluronic L92-PAA Microgel in Capsules

A formulation was prepared as following:

About 30 mg of topotecan were weighed and mixed with 100 mg of the microgel. The mixture was dissolved in 5 mL of 70% ethanol and incubated for 16 hours at room temperature. Then, the half volume of solvent was evaporated in a stream of nitrogen. About 10 mL of 3% acetic acid were added to the mixture. The final mixture was lyophilized until dryness and loaded into the gelatin capsules.

The control topotecan in capsules was prepared as follows:

About 30 mg of topotecan were weighed and loaded in gelatin capsules (3.75 mg/capsule).

The control and formulated topotecan in capsules were orally administered in normal rats at the dose of 15 mg/kg. The blood sample collection, extraction and HPLC analysis were the same as described in example 8.

The results are shown in the following Table 9:

| | | | | Cₘₐₓ | | | AUC |
|---|---|---|---|---|---|---|---|
| | | | | Ratio | | | Ratio |
| | | | Ratio | Oral | | Ratio | Oral |
| | Topotecan | Cₘₐₓ | Cₘₐₓ | Vs | AUC_{0-10 h} | AUC_{0-10 h} | Vs |
| Formulation | form | [ng/mL] | Lac/Car | I.V. | [ng * h/mL] | Lac/Car | I.V. |
| Control | Lactone | 9.8 | 0.8 | 2.0 | 52.8 | 0.6 | 2.0 |
| | Caboxylate | 11.9 | | | 93.2 | | |
| microgel | Lactone | 12.2 | 0.9 | 2.25 | 71.7 | 0.8 | 2.67 |
| | Caboxylate | 13.5 | | | 84.9 | | |

### Example 12

Oral Administration of Topotecan Formulated with Microgel and L92 in Capsules

A formulation was prepared as following:

About 30 mg of topotecan were weighed and mixed with 100 mg of microgel EGDMA and 30 mg of L92. The mixture was dissolved in 5 mL of 70% ethanol and incubated for 16 hours at room temperature. Then, the half volume of solvent was evaporated in a stream of nitrogen. About 10 mL of 3% acetic acid were added to the mixture. The final mixture was lyophilized until dryness and loaded into the gelatin capsules. The topotecan formulation in capsules was orally administered in normal rats at the dose of 15 mg/kg. The blood sample collection, the extraction and HPLC analysis procedures were the same as in example 8.

The results are shown in the following Table:

| | | | | Cₘₐₓ | | | AUC |
|---|---|---|---|---|---|---|---|
| | | | | Ratio | | | Ratio |
| | | | Ratio | Oral | | Ratio | Oral |
| | Topotecan | Cₘₐₓ | Cₘₐₓ | Vs | AUC_{0-10 h} | AUC_{0-10 h} | Vs |
| Formulation | form | [ng/mL] | Lac/Car | I.V. | [ng * h/mL] | Lac/Car | I.V. |
| Microgel + L92 | Lactone | 12.8 | 0.6 | 1.5 | 84.3 | 0.7 | 2.33 |
| | Caboxylate | 19.9 | | | 123.7 | | |

### Example 13

I.V. and Oral Administration of ³H-P85 in Normal Rats

³H-P85 was obtained from PerkinElmer Life Science (Boston, Mass., USA).

Solution of ³H-P85 (160 μCi) was mixed with 5.5 mL of 0.5% solution of cold P85 and i.v. or orally administered in normal rats at the dose of 14 mg/kg. Three animals per group were used.

After various time intervals (15, 30 min, 1, 3, 6, 10 and 24 hrs) post-injection, the blood samples were collected. Plasma was separated by centrifugation at 3000 RPM for 5 minutes and frozen immediately in dry ice.

Plasma samples were defrosted before analysis and hydrogen peroxide (5 μL) was added to each aliquot of plasma samples (80 μL) and incubated over night at 4° C. The plasma aliquots were transferred to scintillation vials, mixed with scintillation liquid (4 mL) and counted on radioactivity counter.

The results are shown in the Table 10:

| | Cₘₐₓ | Cₘₐₓ | AUC0-24 h | AUC_{0-24 h} |
|---|---|---|---|---|
| Compound | [μg/mL] | % of I.V. Adm. | [μg * h/mL] | % of I.V. Adm. |
| ³H-P85 I.V. | 34.7 | 100 | 129.0 | 100 |
| ³H-P85 Oral | 1.5 | 4.5 | 20.9 | 16.2 |

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described compositions and modes for carrying out the invention which are obvious to those skilled in the art or related fields are intended to be within the scope of the following claims.

What is claimed is:

1. A pharmaceutical composition comprising a lactone form of camptothecin, or a lactone form analog thereof that is effective for controlling abnormal cell proliferation; and at least one amphiphilic block copolymer that, upon oral administration to a patient, increases oral bioavailability of the lactone form significantly more than that of a directly related carboxylate form of the camptothecin or analog thereof.

2. A composition according to claim 1 wherein at least one amphiphilic block copolymer is selected from the group consisting of poly(ethylene oxide)-b-poly-propylene oxide; poly(ethylene oxide)-alpha tocopherol; poly-ethylene oxide poly-alkyl; and, poly-ethylene oxide poly-lactide.

3. A composition according to claim 2 comprising the lactone form of camptothecin or a lactone form analog thereof selected from the group consisting of 9-aminocamptothecin; 7-ethylcamptothecin; 10-hydroxycamptothecin; 9-nitrocamptothecin; 10,11-methlyenedioxycamptothecin; 9-amino-10,11-methylenedioxycamptothecin; 9-chloro-10,11-methylenedioxycamptothecin; irinotecan (CPT-11); topotecan; 7-(4-methylpiperazinomethylene)-10,11-ethylenedioxy-20(S)-camptothecin; 7-(4-methylpiperazinomethylene)-10,11-methylenedioxy-20(S)-camptothecin; and, 7-(2-(N-isopropylamino)ethyl)-(20S)-camptothecin.

4. A composition according to claim 3 wherein at least one amphiphilic block copolymer is poly(ethylene oxide)-b-poly(propylene oxide).

5. A composition according to claim 4 wherein at least one amphiphilic block copolymer is a hydrophobic block copolymer.

6. A composition according to claim 4 wherein a poly(oxypropylene) portion of at least one of poly-ethylene oxide poly-propylene oxide block copolymers comprises at least 50% by weight of the block copolymer.

7. A composition according to claim 6 wherein at least one poly-ethylene oxide poly-propylene oxide block copolymer is selected from the group consisting of PLURONIC® P85, L92, L61, P84, L101, L121, P83, and P81.

8. A composition according to claim 7 wherein the poly-ethylene oxide poly-propylene oxide block copolymer is PLURONIC® P85 and a form analog of camptothecin is topotecan or 9-nitro-20(S)-camptothecin.

9. A pharmaceutical composition comprising a lactone form of camptothecin, or a lactone form analog thereof that is effective for controlling abnormal cell proliferation; an amphiphilic block copolymer that increases oral bioavailability of the lactone form, significantly more than a carboxylate form of the camptothecin or analog thereof; and at least one bioadhesive polymer that protects a closed alpha-hydroxy lactone structure of the lactone form of camptothecin or analog thereof, and provides a controlled release of the lactone form of camptothecin or lactone form analog thereof, in GI, upon oral administration to a patient.

10. A composition according to claim 9 wherein at least one bioadhesive polymer is selected from the group consisting of soluble polymer, insoluble polymer, biodegradable polymer, nonbiodegradable polymer, hydrogel polymer, thermoplastic polymer, natural polymer, synthetic polymer, homopolymer, and copolymer, wherein the bioadhesive polymer increases the drug residence time in GI when applied to the mucosal surface of mammalian intestine.

11. A composition according to claim 10 which provides a sustained and controlled release wherein at least one amphiphilic block copolymer is selected from the group consisting of poly-ethylene oxide poly-propylene oxide; poly-ethylene oxide-alpha tocopherol; poly-ethylene oxide poly-alkyl; and, poly-ethylene oxide poly-lactide.

12. A composition according to claim 11 comprising the lactone form of camptothecin or a lactone form analog thereof selected from the group consisting of 9-aminocamptothecin; 7-ethylcamptothecin; 10-hydroxycamptothecin; 9-nitrocamptothecin; 10,11-methlyenedioxycamptothecin; 9-amino-10,11-methylenedioxycamptothecin; 9-chloro-10,11-methylenedioxycamptothecin; irinotecan (CPT-11); topotecan; 7-(4-methylpiperazinomethylene)-10,11-ethylenedioxy-20(S)-camptothecin; 7-(4-methylpiperazinomethylene)-10,11-methylenedioxy-20(S)-camptothecin; and, 7-(2-(N-isopropylamino)ethyl)-(20S)-camptothecin.

13. A composition according to claim 12 wherein at least one bioadhesive polymer is a copolymer and at least one bioadhesive segment of the copolymer is polyacrylic acid (PAA).

14. A composition according to claim 13 wherein at least one bioadhesive polymer is a copolymer of polyacrylic acid (PAA) and a PEO-PPO-PEO block Poloxamer (polyether).

15. A composition according to claim 14 wherein the copolymer is selected from the group consisting of PLURONIC® F127-PAA, L92-PAA, F68-PAA, F88-PAA, F108-PAA, P105-PAA, P103-PAA, L61-PAA, and F38-PAA.

16. A composition according to claim 14 wherein the copolymer is a graft-copolymer wherein the PAA and the polyether are linked to each other by a plurality of C—C bonds.

17. A composition according to claim 16 wherein the copolymer is cross-linked and is selected from the group consisting of F127-PAA-EGDMA, L92-PAA-EGDMA, F68-PAA-EGDMA, F88-PAA-EGDMA, F108-PAA-EGDMA, P105-PAA-EGDMA, P103-PAA-EGDMA, L61-PAA-EGDMA, and F38-PAA-EGDMA.

18. A method for the prevention and/or treatment of a condition related to abnormal cell proliferation comprising orally administering to a patient in need of thereof an effective amount of a composition according to claim 3.

19. A method for the prevention and/or treatment of a condition related to abnormal cell proliferation comprising orally administering to a patient in need of thereof an effective amount of a composition according to claim 12.

20. A method for the prevention and/or treatment of a condition related to abnormal cell proliferation comprising orally administering to a patient in need of thereof an effective amount of a composition according to claim 16.
